(19) United States
(12) Patent Application Publication
(10) Pub. No.: US 2026/0364254 A1
(43) Pub. Date: Nov. 19, 2026

(54) Apparatus and Process for Precision Control of a Fluid in IV Delivery System

(71) Applicant: Safer Infusion Systems, Inc., Newbury Park, CA (US)

(72) Inventor: Thomas G Campbell, Newbury Park, CA (US)

(73) Assignee: Safer Infusion Systems, Inc., Newbury Park, CA (US)

(21) Appl. No.: 19/190,607

(22) Filed: Apr. 26, 2025

(51) Int. Cl. A61M 5/168; A61M 5/14; A61M 39/22; A61M 39/24

(52) U.S. Cl. CPC A61M 5/16881; A61M 5/1414; A61M 39/24; A61M 2039/226; A61M 2202/0007; A61M 2202/04; A61M 2205/3331; A61M 2210/12

(57) ABSTRACT

Control of a fluid in an IV delivery system is achieved through use of a valve body in which a fluid passage between its inlet and outlet is controlled by an actuation mechanism to provide precise, repeatable flow control achieved by movement of a needle plunger relative to a valve seat. Five different embodiments of an actuation mechanism are a rotational needle plunger valve, a linear rack and pinion needle plunger valve, a lever-actuated needle plunger valve, a slot cam drive needle plunger valve, and a crank disc drive needle plunger valve, and a check valve can be integrated at the outlet to prevent backflow of fluid into the fluid passage.

### FIELD OF THE INVENTION

The present invention relates to the field of fluid flow control devices, specifically to liquid control valves designed for low-pressure applications. More particularly, the invention pertains to valve systems and mechanisms for regulating the flow of liquids in environments where operating pressures do not exceed 50 psi in medical fluid delivery systems. The invention encompasses valve configurations employing variable restriction elements to achieve precise modulation of liquid flow through flexible or rigid conduits.

### BACKGROUND OF THE INVENTION

Intravenous (IV) fluid administration relies on precise drip rate control to deliver fluids and medications effectively in medical settings. Roller clamps, commonly used with flexible tubing to regulate flow in IV drip chambers, represent a simple and low-cost solution for this purpose. There are two methods of establishing a flow control region within the clamp housing. With one, the wheel travels along an inclined ramp molded into the bottom wall of the device. The other method involves inclined wheel guides, allowing the wheel to travel at an angle to the base of the clamp. Pressure on the IV tubing varies with the up and down movement control wheel as it cycles along the inclinations in the wheel guides. Some roller clamps incorporate a dual-action feature, allowing both a ramp and a variable, inclinable control wheel to adjust the lumen of the IV tube.

Roller clamps operate in a low-pressure environment, typically below 50 psi. Despite their widespread use, roller clamps exhibit significant technical deficiencies that compromise precision and reliability. Variations in tubing properties—such as material composition, wall thickness tolerances, durometer, compression set, tensile strength and modulus, and tear resistance—introduce inconsistent flow responses, as the tubing's deformation under compression varies unpredictably. Additionally, the molded plastic housing and thumb wheel are subject to manufacturing tolerances and material property variations, including creep and wear, further reducing repeatability and fine control. As a result, roller clamps often yield non-linear and erratic drip rates, challenging their suitability for applications requiring therapeutic accuracy.

A basic review of available valve technologies is presented in the chart on FIG. 1. Here, the basic valve designs are ranked according to the level of precision that they provide. Needle valves stand out as offering the highest level of precision, and pinch valves, under which roller clamps fall, are fourth in line.

The shortcomings of roller clamps prompted the adoption of electronic infusion pumps, which deliver precise flow regulation through automated systems. However, the complexity, high cost, and maintenance demands of infusion pumps render them impractical for routine IV administration or resource-constrained environments. The medical field thus faces a gap between the imprecise simplicity of roller clamps and the expensive sophistication of electronic alternatives. There is a clear and long-felt need for a straightforward, cost-effective device with minimal components that overcomes the material and mechanical limitations of roller clamps to deliver reliable and precise flow control for managing drip rates in IV drip chambers.

Healthcare spending in the U.S. reached $4.8 trillion in 2023, up 7.5% increase from 2022. This translates, on a cost per person basis, to $14,570 according to the Centers for Medicare and Medicaid Services. An opportunity to make inroads with health care cost reduction is to implement a low-cost, precision valve, minimizing requirements in many settings for expensive electronic pumps and their accompanying training, maintenance, and cybersecurity risks, while reducing nurse workload. A precision manual flow controller matches well with a simpler, gravity-fed system when coupled with the burette system as described in U.S. Ser. No. 18/752,035, filed Jun. 24, 2024, the disclosure of which is specifically incorporated herein by reference, all working together to ensure there are minimal to no complications with every PIVC procedure. And further emphasizing reliance on reliable gravity-feed, growing cybersecurity concerns with electronic pumps can affect upwards of 90 percent of patients in a hospital or clinic. IV infusions simply cannot go “offline”. Yet the risk exists, as in 2024 alone, there were two massive attacks: United Health's breach cost them $2.9 billion, and Ascension's breach cost them $1.3 billion.

With two billion IV infusions given worldwide, even the most basic therapy requires a valve of some sort to regulate the drip-rate flow in a drip chamber below any IV infusion bag or bottle. With today's mold making and production molding technology levels, a precision needle valve according to the present invention can be implemented to address cost and risk issues to overcome industry inertia.

### DISCUSSION OF PRIOR ART

Reviewing the history of IV flow control, in particular with liquids in tubing, we have seen a progression of tube pinching hardware starting with metal screw-type clamps, to plastic screw-type clamps, to very simple C-clip (single and multiple position) clamps, and V-notch slider clamps. Two-piece roller clamps steadily evolved to gain broad use in IV therapy, providing a combination of flow adjustment with single-handed operation. As roller clamps typically do not provide demarcations for estimating a flow rate, dial-a-flow systems were developed. These two-section cylindrical dial-a-flow valves apply a twisting closure force on tubing. A more recent design concept employs the use of a bent-leg clamp where the tubing is squeezed between an inner arc and outer arc, applying closure force over a wider length of tubing.

None of these tube-pinching methods delivered high-precision flow control for a host of reasons. First, dimensional tolerance stack-ups between the elastomeric extruded tubing inner and outer dimensions, combined with the dimensional tolerances of molded plastic components, create variation in operation. Second, the elastomeric tubing materials experience variations in durometer, compression set, tensile strength and modulus, and tear resistance.

“Investigating the static or dynamic flexural and compressive stresses on flexible tubing: Comparison of clamp and peristaltic pump impact on surface damages and particles leaching during infusion acts” https://doi.org/10.1016/j.jmbbm.2021.104737 provides an image set shown in FIG. 2 detailing the compression damages on the inner lumen of the tubing, especially in the static condition.

All of these factors interact when tubing is compressed by various means over time. Effectively, they can impact repeatability and consistency of flow control precision. To achieve higher levels of precision flow control, especially for use with IV infusion drip chambers, the elastomer tubing must be removed from the equation.

Issues with tubing were identified as far back as 1991 with U.S. Pat. No. 5,113,904, where a flow control device for administration of intravenous fluid was proposed based upon a variable cross-sectional area valve that facilitated single-handed operation. This patent considered earlier efforts back to U.S. Pat. Nos. 3,785,378, 3,3877,428, 3,880,401, and his own earlier patents U.S. Pat. Nos. 4,294,246 and 4,361,147. All of these share a common ailment, that of relying on two rigid surfaces moving against each other to control a liquid flow. Examining the filing WO 201802424A1 for adjusting a volumetric flow of infusion liquid in conjunction with a drip chamber, we again see reliance on rotating flow channels that are rigid on rigid surfaces. A more recent filing EP3711792A1 by the same entity now incorporates a diaphragm which has a variable cross-section and an invariable length depending on the displacement position of the actuating element.

We see in U.S. Pat. No. 9,903,481 B2 Control Valve, the ball element incorporates an opening surface area of the outlet opening and comprises a gap-shaped first subregion with a small surface area extending in the direction of rotation and a crescent-shaped second subregion with a large surface area extending symmetrically in the axial direction that are arranged one behind the other in the direction of rotation and continuously merge into one another.

In CN111828671B, a V-shaped ball valve is presented that claims to provide better adjusting performance when the opening degree is small. “When the opening is in the middle, the flow channel is in a fan shape, and the flow area is approximately increased by equal percentage along with the increase of the opening; when the opening is large, the flow channel is combined with a fan shape and a rectangle, the speed increasing change of the flow area along with the increase of the opening is small, and the adjusting performance of the ball valve is ensured.”

With US20230264011A1, we see a recent invention to establish a more linear flow through a ball valve with a V-notch addition to the typical round opening in the ball allowing incremental flow at the low end. The tapered or V-notch design does provide a bit more control over the flow rate compared to a standard ball valve, but it doesn't match the precision of a needle valve because the flow path changes more abruptly.

Needle valves are the most precise valve system for liquid flow control; however, most existing designs typically require a minimum of twelve components, proving too costly to produce in extremely high volumes for a consumable market such as IV infusion sets.

Another factor limiting needle valve usage is non-suitability for single-handed operation as is expected with roller clamps. Existing needle valves rely on multiple 360 degree turns, up to 20, of the knob to transit from fully open to fully closed.

In addition to precision, needle valves inherently deliver a more linear flow profile. As reported by Innovyze in their study about different valve constructions FIG. 3A, Ref. https://help2.innovyze.com/infoworkswspro/Content/HTML/WS/a_valves.htm, flow rates across ball, butterfly, gate, needle, and plug valves are presented in FIG. 3B. Needle valves outperform the other valves in linearity over the closed to fully open range.

### SUMMARY OF THE INVENTION

The present invention is generally directed to precision control of a fluid in an IV delivery system through use of a valve body in which a fluid passage between its inlet and outlet is controlled by an actuation mechanism to provide precise, repeatable flow control achieved by movement of a needle plunger relative to a valve seat

Five different embodiments of an actuation mechanism, and their use, in which flow can be adjusted between a fully open position and a fully closed position in a single-handed operation, are described.

First, a threaded body is rotationally driven by a horizontally oriented thumb wheel in which rotation of the thumb wheel translates the needle plunger. The height of the thumb wheel relative to the valve body can remain constant and change of flow from fully open to fully closed can be achieved, in a single-handed operation, by rotation of the thumb wheel of approximately one-hundred eighty degrees or less. A threaded interface can provide micro-adjustments of the needle plunger with a resolution of less than 0.1 mL/min and a locking mechanism (such as a friction pad), can lock the thumb wheel in place during its operation.

Second, linear movement of a linear rack and pinion system translates the needle plunger to provide precise flow control. Variable gearing can be used in which rack and pinion gear have smaller teeth that graduate to larger teeth at an open end of travel such that very fine plunger control is established at very low flow rates and that near to the open end of travel, the needle plunger advances to fully open more rapidly.

Third, a lever actuated mechanism, which is pivotally coupled to the valve body and operatively connected to the needle plunger, and which can be locked, can be used to translate the needle plunger so as to provide precise flow control.

Fourth, a slot cam drive mechanism can be coupled to the needle plunger so that rotating the slot cam drive forces a pin riding in the slot of the slot cam to guide an attached needle plunger to provide customizable flow control.

Fifth, a crank disc drive mechanism can be operatively coupled to the needle plunger so that rotation of the crank disc translates to upward and downward motion of the needle valve plunger to provide precise flow control. The crank disc rotates vertically on a fixed shaft, with a connecting rod attached via a crank pin at a radius from the center to cycle the needle plunger up and down, and the centroid of the crank disc can either be aligned or offset with the axis of plunger travel.

For each of the five different embodiments of an actuation mechanism, a check valve can be integrated at the outlet to prevent backflow of fluid into the fluid passage.

Accordingly, it is a primary object of the present invention to provide low-cost, precision fluid control devices useful for regulating the flow of fluids in an IV infusion system in a single-handed operation.

This and further objects and advantages will be apparent to those skilled in the art in connection with the detailed description of the invention set forth below.

### BRIEF DESCRIPTION OF THE DRAWINGS

FIG. 1 is a chart of basic valve designs.

FIG. 2 is an image taken from a publication detailing compression damages on the inner lumen of tubing.

FIG. 3A is a chart from a publication about different valve constructions.

FIG. 3B is a graph from a publication about flow rates for various valves.

FIG. 4 illustrates various interconnects of tubing to inflow and outflow sides of a valve body.

FIG. 5 illustrates commonalities of a valve body, needle plunger and actuation mechanism of the present invention.

FIG. 6A illustrates a conventional prior art roller clamp attached to a tubing from an IV bag while FIG. 6B illustrates an apparatus according to the present invention attached to a tubing from an IV bag with a check valve integrated at the outlet of the valve body. FIGS. 6C-E compare the two devices of FIGS. 6A and 6B at three different flow rates.

FIGS. 7A-7D illustrate one rotational needle plunger valve according to the present invention in which FIG. 7A is a top view, FIG. 7B is a cutaway view of FIG. 7A,

FIG. 7C is a side view and FIG. 7D is a bottom view. FIGS. 8A-98 illustrate various flow rates through the cutaway view illustrated in FIG. 7B.

FIGS. 9A and 9B illustrate a rotational needle plunger valve according to the present invention where intermeshing gears are included in the valve and the needle plunger is in a fully open position in FIG. 9A and a fully closed position in FIG. 9B; FIGS. 10A and 10B illustrate the gear set. FIG. 11 is a partial cutaway illustrating movement of the plunger which is further illustrated in FIGS. 12A and 12B.

FIG. 13 illustrates a linear rack and pinion needle plunger valve according to the present invention while FIGS. 14A-C show various positions of the plunger valve and FIGS. 15A-D illustrate how the shape of the needle plunger can affect flow.

FIG. 16 illustrates a lever-actuated needle plunger valve according to the present invention.

FIGS. 17A-17C illustrate how a slot cam is used in the present invention to raise the needle plunger from fully open in FIG. 17A to fully closed in FIG. 17C.

FIG. 18A illustrates a slot cam with a spur gear while FIGS. 18B and 18C illustrate a thumb wheel with a spur gear all on the mating spur gear attached to the slot cam.

FIGS. 18D-18F illustrates how a slot cam is driven by a rotationally fixed thumb wheel to achieve a fully open condition in FIG. 18D to a fully closed condition in FIG. 18F for the needle plunger.

FIGS. 19A-B illustrate a crank disc drive needle plunger valve according to the present invention.

FIG. 20 provides a component count summary of the needle valve design types.

FIG. 21A illustrates a duckbill check valve element captured by a two-piece housing.

FIG. 21B illustrates how the duckbill check valve functions to allow liquid flow in one direction.

FIGS. 21C and 21D present duckbill check valve images with FIG. 21C as a cross-sectional view and FIG. 21D as a three-dimensional image.

FIG. 22A illustrates an umbrella check valve element captured by a two-piece housing.

FIG. 22B illustrates how the umbrella check valve functions to allow liquid flow in one direction.

FIG. 22C is a three-dimensional rendering of an umbrella check valve element.

FIG. 23A shows a dome check valve element captured by a two-piece housing.

FIG. 23B illustrates how the dome check valve functions to allow liquid flow in one direction.

FIGS. 23C and 23D present dome check valve images with FIG. 23C as a cross-sectional view and FIG. 23D as a three-dimensional image.

FIG. 24A illustrates the typical size ranges and relational setup of a roller clamp with a following discrete check valve to prevent backflow through the roller clamp.

FIG. 24B presents a needle valve system with a rack and pinion actuation with an integral check valve as a compact comparison to the system of FIG. 24A.

FIG. 25 lists the cracking pressures, materials of construction, and cost issues for three types of check valves that are most common for volume applications.

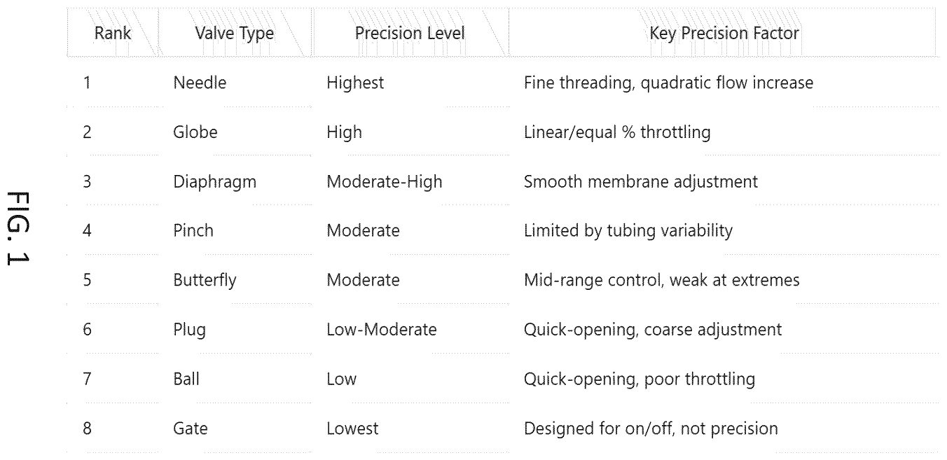

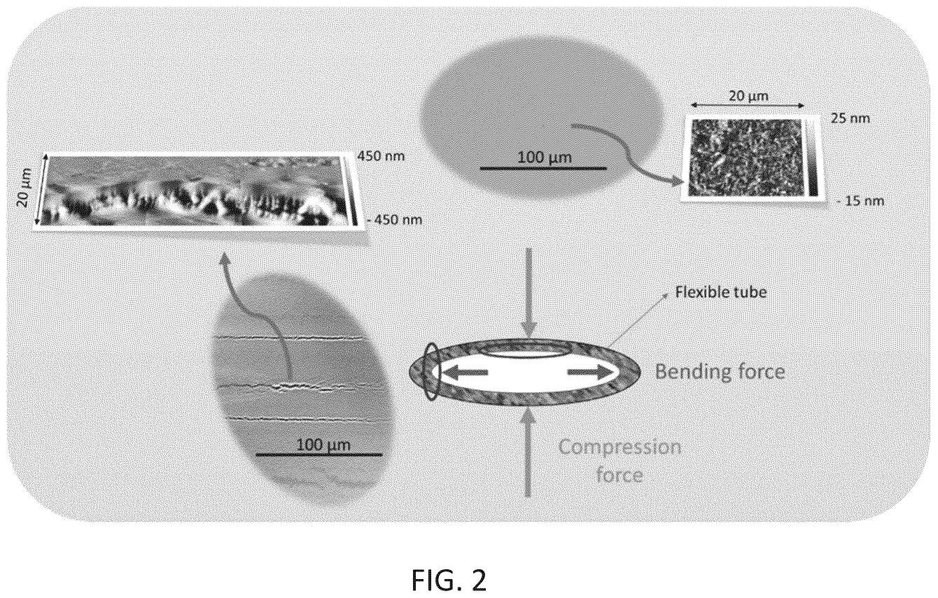

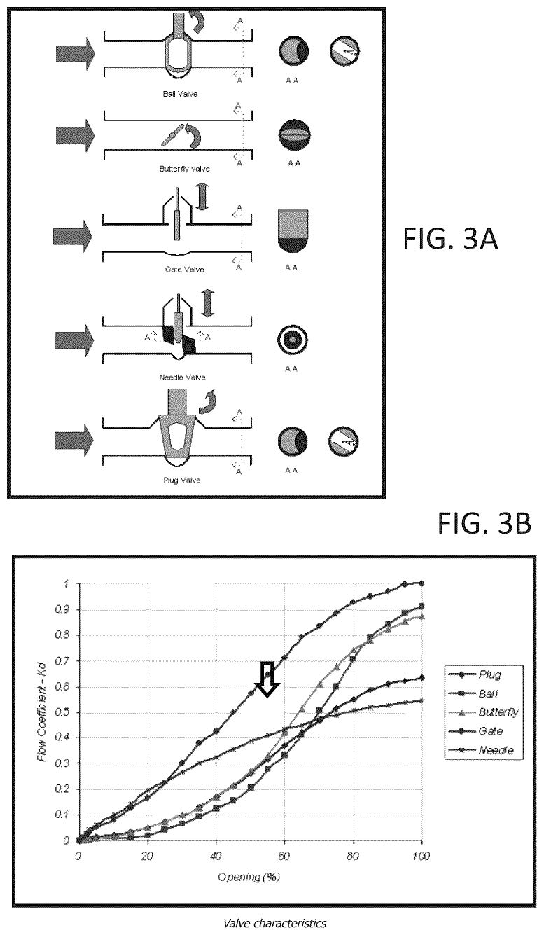

### DETAILED DESCRIPTION OF THE INVENTION

The invention set described herein includes multiple methodologies for mechanical operation of a simple, single-handed control low-pressure valve capable of precise flow control with rapid adjustment from fully closed to fully open. The invention is targeted at replacing single-hand operated roller clamps applied to flex tubing that have proven to be less reliable than desired for consistency and precision flow control. Furthermore, roller clamps do not offer a locking mechanism. Once these precision valves are deployed, we look to have the ISO 8536-13:2024 document amended as its current lax requirements have enabled the proliferation of roller clamps that are questionable in their performance.

Although roller clamps represent a very low-cost option, their implementation leads to higher costs in use because the medical community has widely migrated to electronic infusion pumps to ensure precision flow control. By delivering a simple, relatively low-cost needle valve to complement gravity-fed infusions, system costs can be considerably reduced while maximizing vein patency. With IV infusions, vein patency can be impacted by micropistoning of the catheter in a vein caused by the peristaltic action of an electronic infusion pump. By smoothing out IV fluid drip rates with an all-gravity system, the state of the catheter in a vein will be more stable and perform to the fullest extent of its fluid delivery design life without unnecessary disruption to the patient receiving the IV therapy. It is important to understand that these valve types are incorporated as part of a closed system, once sterilized and packaged, they will protect the infusion process from external contamination and prevent any liquid from escaping.

The heart of the system is a modification to a needle valve where the needle plunger can rotate up and down or move only vertically. With vertical motion, there is an opportunity to take advantage of complex geometries between the plunger and its valve seat, such that at one end of plunger travel, the flow rate can be finely controlled and transition to fully open rapidly at the other end. In one embodiment, the gear rack travels up and down controlled by a thumb wheel similar to that used in a roller clamp, but the thumb wheel has a reducer gear that interfaces with the rack gear such that with less than 180-degree rotation, the plunger can make a fully open to fully closed operation.

With the invention here, both the orifice size and the plunger geometry can establish extreme precision for very low flow infusion rates and transition to a fully open condition. A fully open setting allows a caregiver to flush a line or to quickly fill a burette with saline to a prescribed level. When using the valve to modulate drip rates in a drip chamber, the volumetric flows are small, ranging from a macro drip nozzle where the delivery rate is 10 to 20 drops per milliliter (gtt/mL) with 10, 15 or 20 being the most commonly prescribed. For common drop factors: 10, 15, or 20 gtt/mL are used for rapid infusion of large volumes.

Micro drip nozzles deliver approximately 60 drops per milliliter (gtt/mL) and are used for administering potent medications or for patients sensitive to large doses, such as neonates and pediatric patients. The difference in drip rates between macro and micro nozzles is related to the diameter of the nozzle where the drop enters the drip chamber. The valve directly below the drip chamber is responsible for setting the drip rate in the drip chamber.

The heart of the system is a modification to a needle valve where the needle plunger can rotate up and down or move only vertically. With a vertical motion, there is opportunity for a complex geometry between the plunger and its cavity such that at one end the flow rate can be very low and achieve fully open rapidly at the high end. In one embodiment the gear rack travels up and down controlled by a thumb wheel like on a roller clamp, but the thumb wheel has a reducer gear that interfaces with the rack gear such that with less than a 180-degree rotation, the plunger can make a fully open to fully closed operation.

### DEFINITION OF TERMS

At the core of this invention is a valve element that is technically described as a valve obturator. Valve obturators are categorized into four types based on their movement and design. Our needle valve system is a Closing Obturator where the closure member moves along the seat axis, towards or away from the valve seat, and includes globe, needle, and piston valves. For comparison, with Sliding Obturators, the closure member moves perpendicular to the flow direction and includes gate, ball, plug, and butterfly valves. And although a design option incorporates a needle valve obturator that rotates, it is not a Rotary Obturator. This designation includes gate, ball, plug, and butterfly valves where the closure member has a port that turns through 90 degrees to align with or block the flow direction. And lastly, the category of Flexible Obturator includes diaphragm and pinch valves where the closure member is a flexible passage that is flattened to restrict flow.

Going forward, the needle valve will be termed a closing obturator where the closing member is called a plunger. Depending on the actuation method, this plunger may rotate as it moves up and down against the valve seat, or the plunger may remain in a fixed orientation as it moves up and down against the valve seat.

Currently, pinch valves acting on flexible tubing are the most common valve system for IV infusions outside of the mechanisms employed internally with electronic infusion pumps.

The sliding gear rack and rotational thumb wheel driver can be designed with a range of materials, gear ratios, and size considerations. Typically attached to the thumb wheel is a reducer gear that interfaces with the rack gear to provide a linear motion with precise control.

A cantilever actuator arm is a rigid arm anchored at the base and free at the other end, acting as a lever to transmit force and motion to raise and lower the valve plunger. A cantilever actuator arm relies upon an anchored base. This fixed pivot is the stationary point where the cantilever arm is anchored, allowing it to pivot.

The lever-actuated valve requires a linkage to the valve plunger. The connecting link of the cantilever arm to the plunger transfers motion from the arm to the plunger with a pin coupling mechanism.

A needle plunger is the movable component that travels vertically along a channel to open or close the valve. The design of the needle plunger involves material choices and physical shapes to optimize the fit with the valve seat to manage liquid and air flow with precision.

A plunger stem guide is a structure that constrains the plunger's motion, ensuring linear travel toward or away from the valve seat.

The cylindrical passage in the valve body is referred to as the valve bore. Needle valves feature a tapered, needle-like stem or plunger that mates with a single, precisely machined orifice or valve seat.

The action of raising the cantilever arm to pull the plunger upward, increasing the flow volume is called lift actuation.

The action of lowering the cantilever arm to drive the plunger downward onto the valve seat, sealing it shut, is called closure actuation.

The stationary surface against which the plunger seals to stop liquid flow is called the valve seat. For designs where the needle valve rotates, the valve seat becomes a conical seat. For a plunger that does not rotate, but raises and lowers linearly, the valve seat may be other than conical to optimize laminar flow of liquid or air through small opening gaps between the valve seat and the plunger tip.

The valve cavity includes the opening that allows inflow and outflow of liquids or gases as well as the complex geometry for the valve seat and its total volume when the valve is fully open.

A slot cam is a rotating cam with an arc slot in which a pin from the needle valve shaft rides. As the cam rotates, it drives the mating in an upward or downward direction that, in turn, drives the needle valve plunger up and down.

A cantilever valve assembly includes a cantilever lever anchored at a fixed pivot on the valve body, with a mechanical linkage connecting the free end of the lever to a valve plunger. The plunger moves within a plunger stem guide such that lift actuation of the lever opens the valve, and closure actuation lowers the needle plunger to engage the seat, closing the valve. The stem guide is sealed by a pressure fit between the plunger shaft and the shaft opening in the valve body or by an O-ring situated around the plunger shaft. The O-ring may be held in a fixed location by means of a retaining slot in the valve body, or the O-ring may be locked to the plunger shaft and ride up and down inside of the plunger stem guide. With a design where the O-ring moves, the potential for liquid residue remaining in the valve body is a concern.

### General Description

Described herein are five valve designs: 1) Rotational needle plunger valve, 2) Linear rack and pinion needle plunger valve, 3) Lever-actuated needle plunger valve, 4) Slot cam-actuated needle plunger valve, and 5) crank disc drive needle plunger valve. The function and construction of each design is detailed along with a review of material options. Following the materials and design reviews is an analysis of the applicable flow equations that apply to calculating flows through needle valves. The designs share several commonalities.

They all incorporate a multi-piece housing to which flexible or rigid tubing attaches for inflow and outflow. The housing components are molded from various plastics. Medical grade Polycarbonate (PC) and Acrylonitrile Butadiene Styrene (ABS) both offer good strength and low cost. To handle more chemically aggressive fluids, Cyclic Olefin Copolymer (COC) or Cyclic Olefin Polymer (COP) and Polyetheretherketone (PEEK) are options, with COC and COP, both clear, and PEEK being opaque, which may be preferable for handling light-sensitive liquids. All of these material choices deliver biocompatibility performance and meet USP Class VI or ISO 10993 for medical use. This is important knowing that IV fluids will contact these materials. All of these materials are also sterilizable. PC and ABS are strong choices for maintaining tight tolerances required with snap or press fits and multipart mating, where adhesives or hardware may be employed for assembly.

The criticality of the needle plunger performance represents a materials challenge. For optimum sealing performance, the tip may benefit from a conformal plastic with the upper portion providing rigidity for precise control. Here, a two-material or overmolding approach can work well. The same chemical resistance applies to the plunger as the molded body components. Options for the soft, conformal tip include Thermoplastic Elastomers (TPE) USP Class VI medical grade, such as Medalist (Teknor Apex) or Versaflex (Avient), Silicone ((Liquid Silicone Rubber-LSR) like medical-grade Dow Corning Silastic or Momentive Silopren, or Polyurethane (TPU) like medical-grade Tecothane (Lubrizol).

The upper portion of the needle plunger should overmold well and may be PC for strength and toughness or PEEK for chemical stability. However, adhesives or special processing may be required for suitable bond interfaces between rigid and semi-flexible materials. Ultimately, the plunger must meet USP Class VI or ISO 10993 standards for IV fluid contact.

With sufficiently tight molding tolerances and careful assembly, a liquid-tight interface where the plunger shaft transits up and down in the housing opening to the liquid chamber may be achievable as the number of open and close cycles for a disposable medical device may only number from 1 to 100 at most. In production, especially with very high volumes, the tolerances of components may not provide absolute liquid-tightness. In this case, an O-ring will be necessary for liquid-tightness around the plunger shaft as it moves to raise and lower the needle plunger tip. Embedded into a receptacle slot in the molded housing, an O-ring applies compression sealing to the needle plunger shaft. Although relatively inexpensive, the O-ring does represent an additional assembly step, so if tolerances can be maintained sufficiently high, leak-free valve operation without an O-ring is a production tooling goal.

To ensure a reliable seal, compatibility with IV fluids, including toxic ones, and durability under plunger motion, the O-ring must maintain elasticity and resist wear from the plunger's mechanical action. Material choices include medical-grade Silicon Rubber (VMQ), Fluorocarbon Rubber (FKM, e.g., Viton®), Ethylene Propylene Diene Monomer (EPDM), Nitrile Rubber (NBR), or Perfluoroelastomer (FFKM, e.g., Kalrez®). For general IV fluids Silicone Rubber or EPDM will be most cost-effective and biocompatible for water-based fluids. For aggressive IV fluids (e.g., Chemotherapy Drugs, Solvents), FKM (Viton) or FFKM (Kalrez) offer superior chemical resistance. For Lipid-Based IV Fluids (e.g., Parenteral Nutrition), NBR or FKM are better suited to oils and emulsions. Again, all materials must meet USP Class VI or ISO 10993 standards for IV fluid contact.

Within the O-ring material type, an important specification is the Durometer (Hardness). Typically, 50-70 Shore A is suitable for sealing without excessive friction. Softer O-rings (e.g., 50A silicone) improve conformability, while harder ones (e.g., 70A FKM) enhance durability. The groove design, O-ring diameter, and thickness must be perfectly sized to avoid over-compression and leakage.

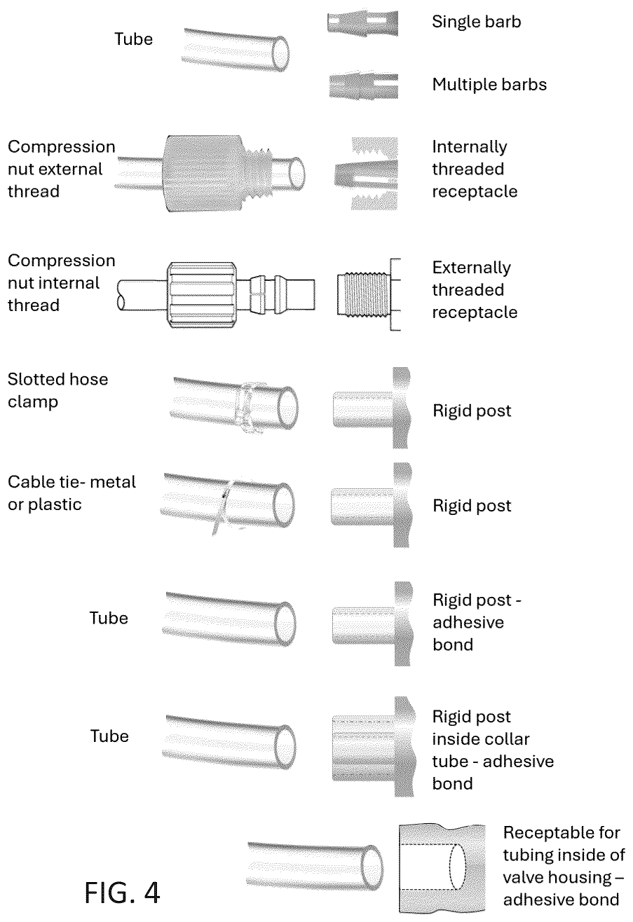

In eliminating the flex tubing from the valve design as is inherent with a roller clamp, we have the requirement to interconnect tubing to both the inflow and outflow side of the valve body. Interconnection of flexible or rigid tubing is accomplished by having the tubing mate with a precision valve by any of the four methods described in FIG. 4. The simplest, an expansion fit over a barb fitting, may be enhanced for a higher retention force with adhesive. It is also possible to forgo the barb element for molding simplicity and employ a tube and adhesive on the outer surface of the tube protruding from the valve body. Or the adhesive can be deployed on the inside of the protruding tube, and the flex tubing will slide inside of the receptacle tube. Field installable options include an external clamp or a separate hollow threaded interface for compression of an inner surface against the tubing outer wall. A mechanical clamp may compress the flex tubing to an external rigid post, although this is cumbersome in a compact design. A preferred field installable option is with an internally threaded receptable or an externally threaded post onto which a compression nut twists onto apply a liquid-tight sealing force. A collar system with one to two pieces may slide over the outside of the flex tubing to affect a proper compression seal.

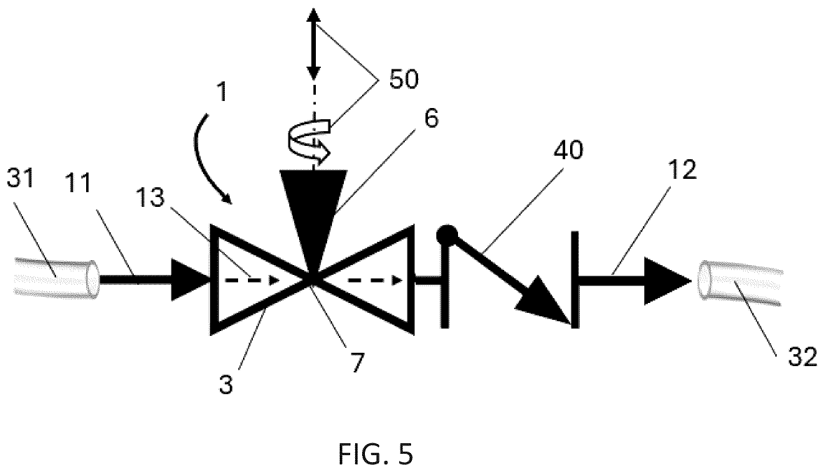

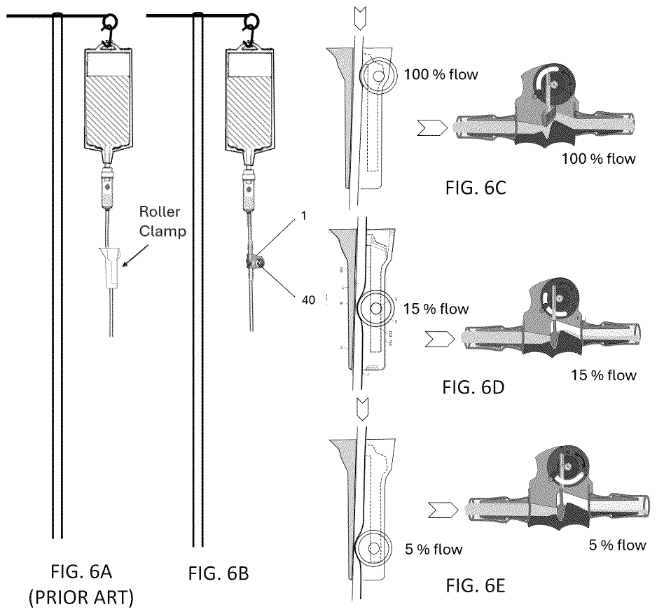

FIG. 5 illustrates the commonalities in a functional format. Apparatus 1 for precision control of a fluid in a delivery system has a valve body 3, an inlet 11, an outlet 12, and a fluid passage 13 connecting the inlet to the outlet. A needle plunger 6 and a valve seat 7 are configured within valve body 3, wherein movement of needle plunger 6 relative to valve seat 7 controls fluid flow within fluid passage 13 from a fully open position to a fully closed position. An actuation mechanism 50 for controlling movement of needle plunger 6, configured to convert a user input into plunger motion, enables flow rates in an environment where an operating pressure does not exceed 50 psi, and actuation mechanism 50 is configured for single-handed operation. A first flexible tubing 31 can be connected to inlet 11 and a second flexible tubing 32 can be connected either to outlet 12 or to a check valve 40 integrated at outlet 12.

Given this summary of commonalities, we can now move to the mechanical operative method for each of the five especially preferred designs.

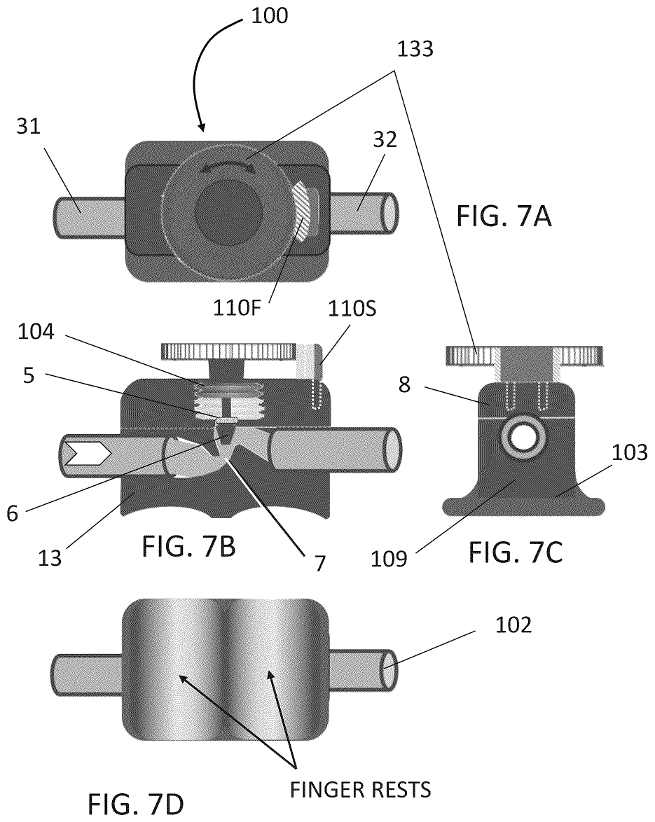

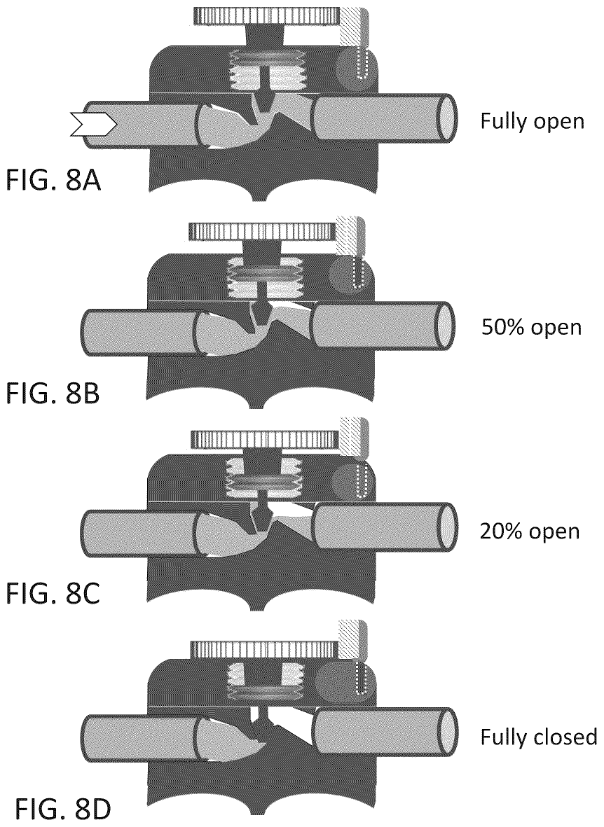

First is the Rotational Needle Plunger Valve, generally designated as 100, that utilizes a horizontal top thumb wheel (133). In FIGS. 7A-7C, the top thumb wheel (133) connects directly to a large diameter threaded body (104) that travels up and down against the fixed threads inside of the valve body (103). Attached to the bottom of the threaded body is the needle plunger (6). The shaft of the needle plunger slides through an opening in the valve body, with or without an O-ring (5). The rotating needle plunger moves down to fully seal against the valve seat (7). This design typically requires three full rotations of the thumb wheel to transit from fully open to fully closed, which is less than ideal for single-handed operation and the thumb wheel will rise an equivalent height of the travel of the large diameter threaded body (104)

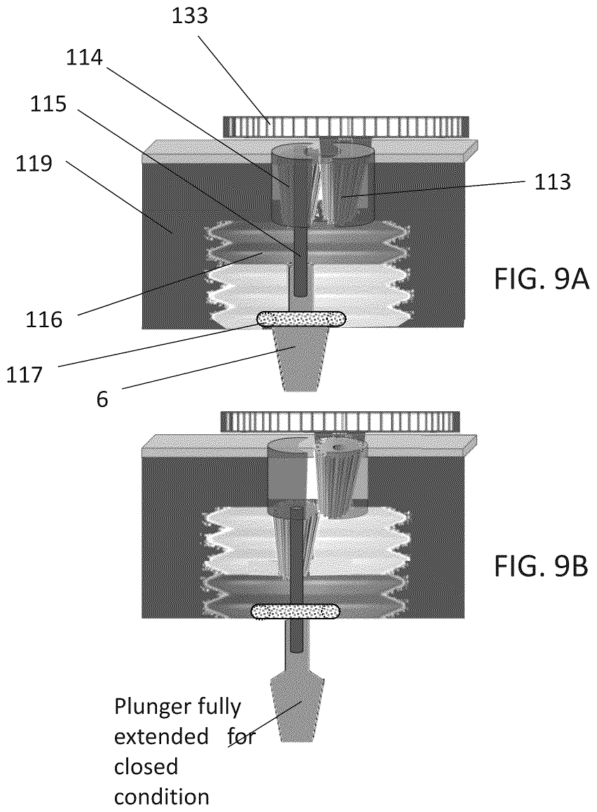

Presented in FIGS. 8A-D are liquid flow pattern diagrams showing fully closed, 20 percent open, 50 percent open, and fully open. In FIGS. 9A and 9B, thumb wheel 133 is offset from the plunger path, and the thumb wheel does not rise above the valve body. Attached to thumb wheel 133 is an elongated spur gear (113). The thumbwheel/elongated spur gear rotates on a fixed axis. Meshing with this elongated spur gear is another elongated spur gear (114) that is attached to a large diameter threaded body (116), as with the previous design, but the two elongated gears provide three complete turns of the needle valve with only a single turn of the thumb wheel through the gear ratio. As the large diameter threaded body lowers in the valve body to seal the valve plunger against the valve seat, the second elongated spur gear (114) rides downward against the first elongated spur gear (113), retained in place by a hollow column that maintains the vertical orientation of the second elongated gear.

In both designs, a friction pad 110F or a resilient single or multi-tooth spring element (110S) presses against the knurls of the thumb wheel creating a retention force to hold the thumb wheel in a specific position. The thumb wheel (133) knurls also provide tactile feedback for precision operation of the needle valve.

A further enhancement to the Rotational Needle Plunger Valve is the incorporation of four intermeshing gears as shown in FIGS. 10A and 10B. Thumb wheel (133) (Gear D) has teeth on its perimeter that engage with a small gear (120) atop a larger diameter gear (115) (Gear B). This gear engages with the spur gear (122) (Gear A) that is directly attached to the needle valve plunger shaft (18). With approximately 180 degrees of rotation of the thumb wheel (133) we can achieve 3 full rotations of the spur gear (122) to move the needle plunger (6) from fully closed to fully open. FIG. 11 presents a cross-sectional view of the gear set with an indication that the needle plunger can travel 3 to 4 millimeters in vertical distance from the valve seat to a fully open flow condition. Thumb wheel (133) might have an exposed section (125) that allows easy single-handed operation of this rotational needle valve system. FIGS. 12A and 12B illustrate movement of needle plunger 6.

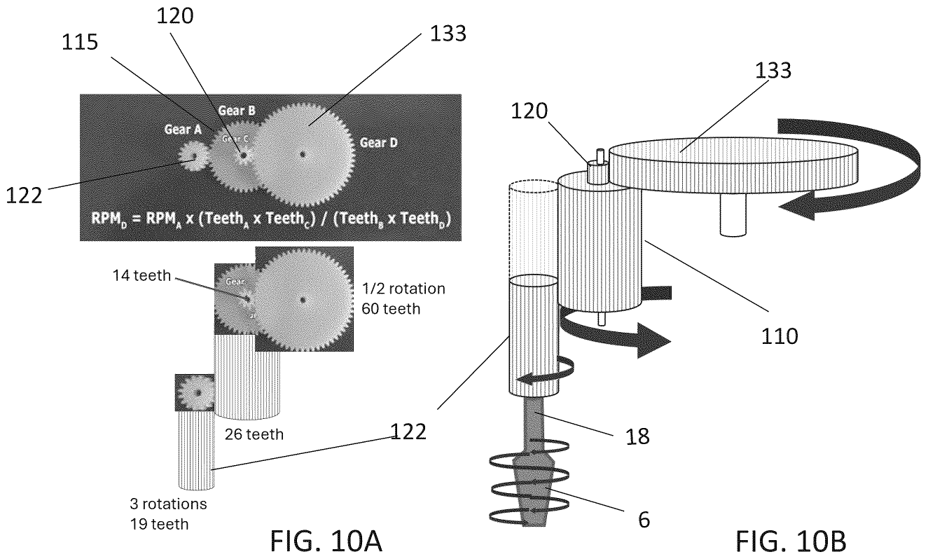

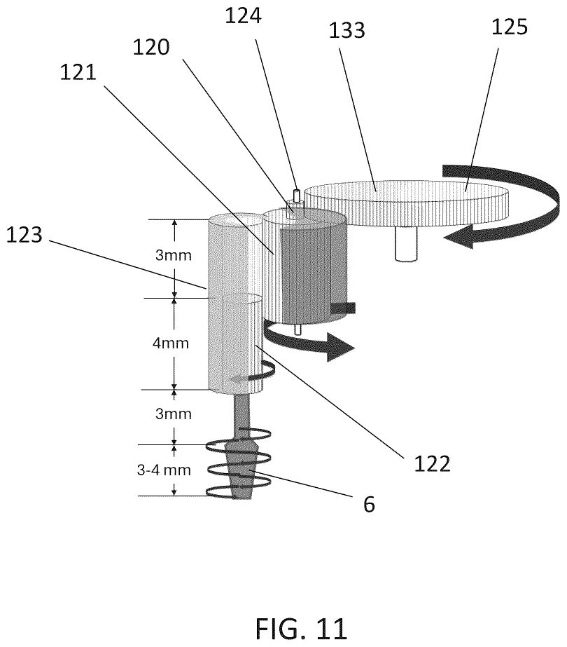

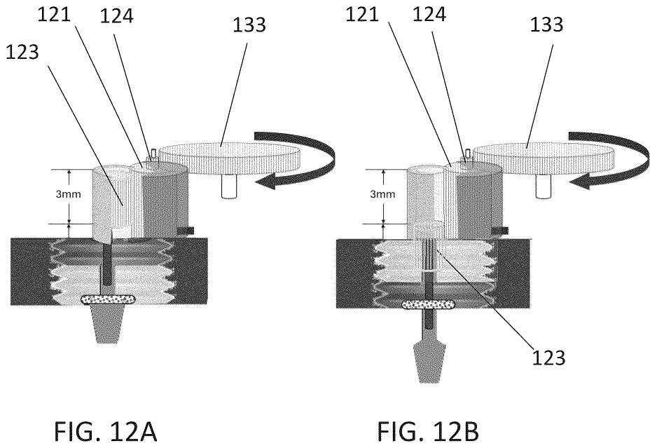

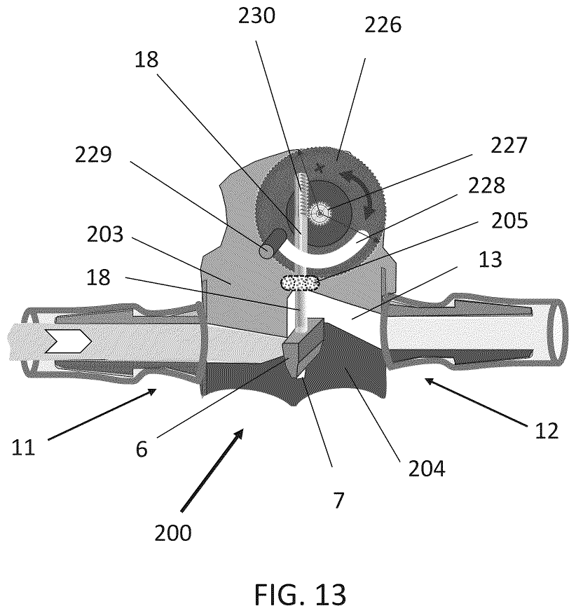

The second design implements a linear rack and pinion needle plunger valve as shown in FIG. 13, generally designated 200. A thumb wheel (226) is aligned with the axis of inlet 11 and output 12, like a roller clamp design. The thumb wheel either has a post perpendicular to the wheel body or a hole through which a retaining pin fits to mount the thumb wheel and allow it to rotate. Attached to the knurled thumb wheel is a small pinion gear (227) that interfaces with a moving rack (230). The needle valve shaft (18) and needle plunger (6) are attached to and in line with the geared rack (230). As the thumb wheel (226) is rotated through approximately 180 degrees, the rack (230) and, in turn, the needle plunger (6) can cycle from fully open to fully closed positions. A variable gear ratio may be employed with this rack (230) and pinion (227) interface such that a smaller tooth size is used at the top of the rack (230) transitioning to larger tooth dimensions at the bottom section of the rack (230). With this configuration, when the rack is fully extended in the closed valve position, the smaller gear teeth allow for a finer level of control as the needle valve plunger just starts to open, which is the critical zone for setting a drip flow rate with an associated IV fluid drip chamber. Towards the bottom of the rack, the gear teeth are larger, allowing a more rapid advancement to fully open, all within the approximately 180-degree rotation of the thumb wheel. As illustrated in FIG. 14A-C, three positions of the thumb wheel (226) incorporating an arc slot (228) show fully closed, 30 percent open, and fully open positions of the needle valve. A tapered pin (229) rides in this arc slot. When the thumb wheel (226) is stopped at a desired position, the tapered pin (229) can be pushed laterally, with a friction fit against the arc slot (228) inner sides. By having the pin (229) extend beyond the valve body 203 in its un-activated position, it can then extend out of the other side of the valve body when in the activated position. By toggling the pin (229) from side to side, it can lock and unlock the desired position of the thumb wheel (226) to reliably hold that flow rate position. This pin (229) may be colorized to visually ascertain if the pin (229) is out, say blue, or if the pin (229) is locked and the extended, exposed section of the pin is red.

With the needle valve plunger 200 only traveling in the vertical axis, we have the opportunity to exercise a range of plunger shapes to finely manage the liquid flow pattern. A unique plunger shape shown in FIGS. 15A-D demonstrates how a wedge design can have a varying relationship with the valve seat, creating well-controlled laminar flow at low flow rates.

While variable pitch racks and pinions can be produced, they do pose some challenges in manufacturing and design. Their complex geometry requires precise engineering to ensure smooth engagement between the rack and pinion. However, with today's mold-making capabilities, mass production with the necessary precision can be achieved.

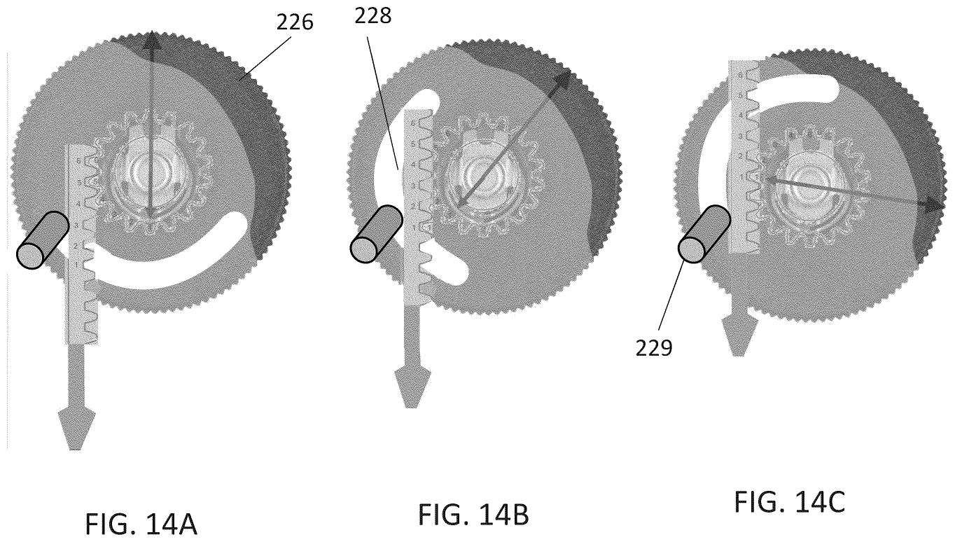

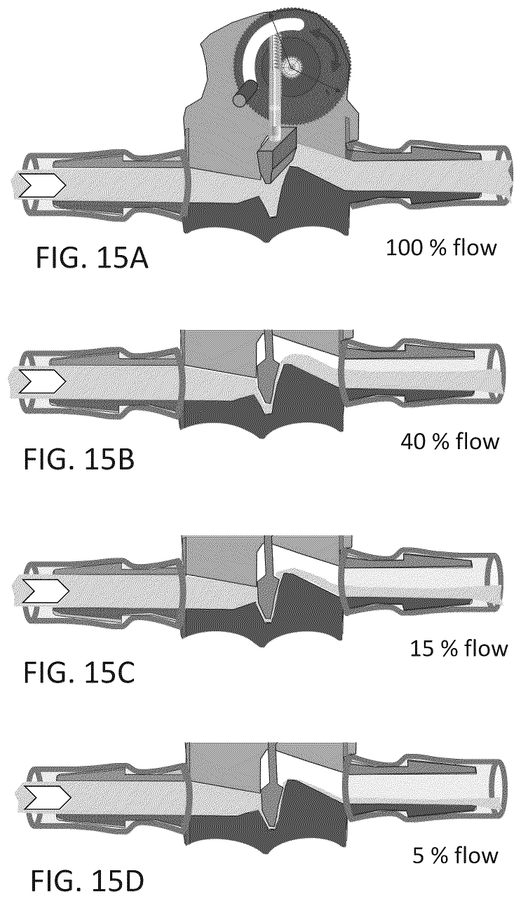

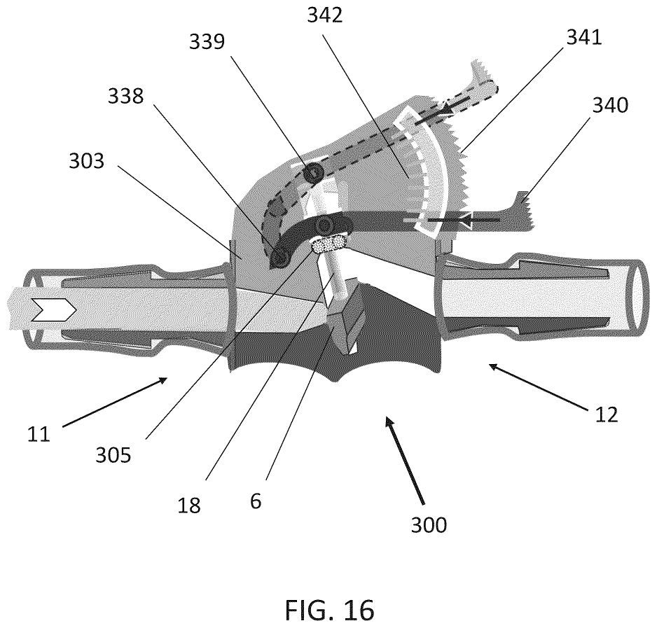

The third design employs a lever-actuated needle plunger valve for precision flow control. The valve assembly shown in FIG. 16, generally designated 300, includes a cantilever lever (340) anchored at a fixed pivot (338) on the valve body (303), with a mechanical linkage (339) connecting the free end of the lever (340) to a valve plunger (6). The plunger moves within a plunger stem guide and O-ring (305) such that lift actuation of the lever (340) opens the valve, and closure actuation lowers the needle plunger (6) to engage with valve seat (7), closing the valve. The pivot point (338) is designed to withstand mechanical forces during operation.

The connection between the lever (340) and the shaft of a needle valve plunger (18) must allow for precise control of the plunger's linear motion (in and out) while the lever pivots around its anchor point (338). Minimum play or slippage is required for each connection option for attaching the lever to the needle valve plunger shaft. A Pinned Joint (Clevis or Hinge Connection) employs a pin that passes through a hole in the lever (340) and a clevis or forked end attached to the plunger shaft (18), creating a pivoting joint. This design is simple, reliable, and allows rotational freedom while transferring smooth, linear motion.

FIG. 15 also shows a set of indicators (342) that may be printed onto the upper valve body or be provided as part of the mold tooling. A marker on the level arm will align with a specific marker on the valve body to indicate the fully open or percent closed position of the valve plunger.

A Ball-and-Socket Joint employs a ball on plunger shaft (18) that fits into a socket on the lever (340). If the ball of the plunger can be press-fit into plunger shaft (18), this might simplify the assembly process.

A Slot Connection is where the lever (340) has a slot that engages a pin or roller attached to plunger shaft (18), allowing the pin to slide along the slot as the lever (340) pivots. This design compensates for the arc of the lever (340), reducing lateral stress on the plunger, and is simple to manufacture. And, as the expected number of cycles is low, slot wear is not a concern. If there is a design requirement for a greater arc of motion of the lever, the sliding slot connection is more suitable.

With a Threaded or Clamped Collar, the lever (340) is attached to a collar or sleeve that clamps or pins onto the plunger shaft (18), often with a pivot point on the collar. Although this option would provide minimal play in the needle plunger control, adding more components is not necessarily a design goal.

A Cam or Eccentric Mechanism can be utilized by attaching the lever arm (340) to a cam or eccentric disc that presses against the plunger shaft (18) or a follower attached to it. This option allows non-linear control of plunger movement. Overall, its increased complexity with design and manufacturing may outweigh its non-linear control benefits.

For fine control (e.g., in IV therapy needle valves), pinned joints or clamped collars minimize play. Compact systems may favor simple pinned or slotted designs over bulkier linkages.

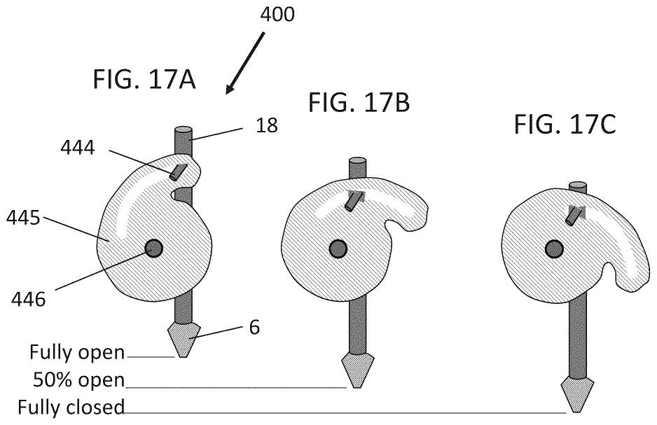

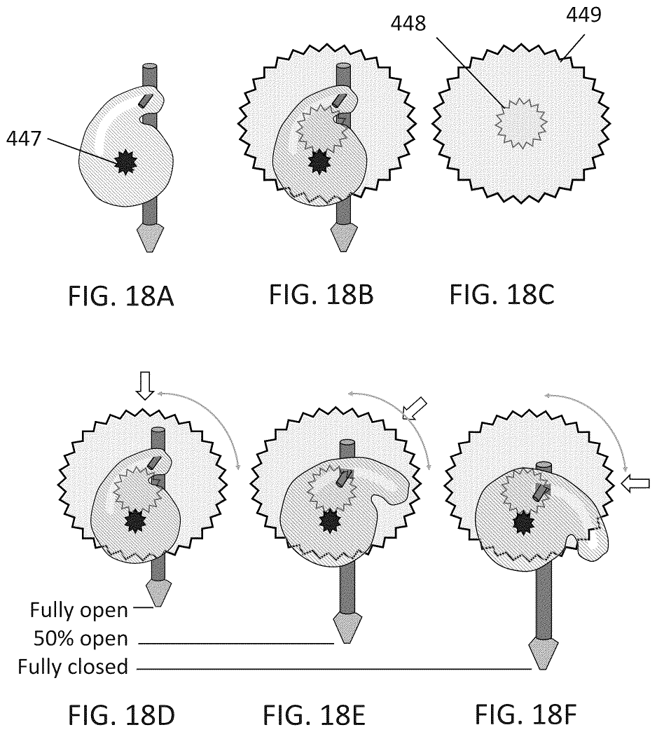

A fourth design employs a slot cam that drives the needle valve plunger up and down as illustrated in FIGS. 17A-C, generally designated 400. A pin on the needle plunger shaft (18) rides inside of the slot of the slot cam (445). Shown in FIGS. 18A-C and 18D-F, the centroid of the slot cam (445) has a gear wheel (447) that engages with a small gear wheel (448) that is at the centroid of the knurled thumb wheel (449). With this design, the length of travel of the needle valve plunger is determined by the length of the slot in the slot cam (445). Although quite simple, this design does not allow a more precise movement of the needle valve plunger at the low flow end of travel, as the movement is only controlled by the shape of the arc slot.

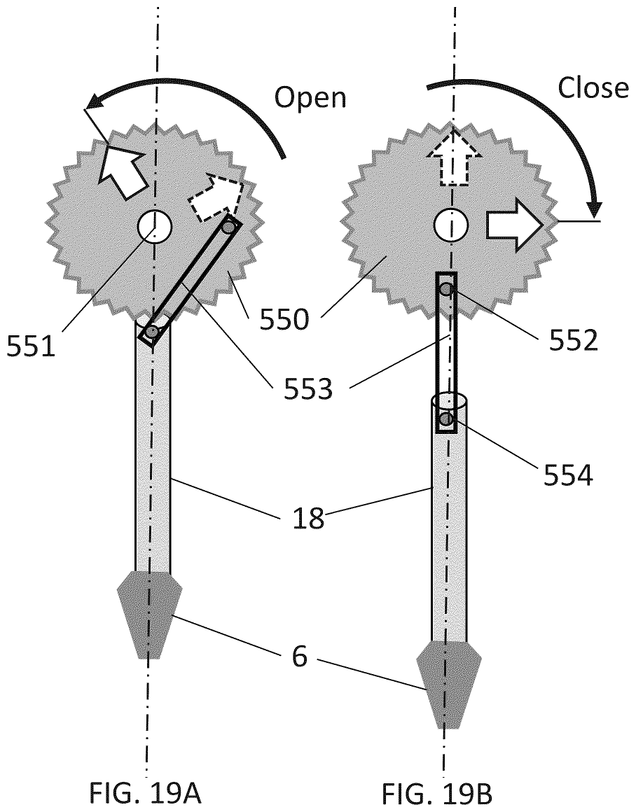

The fifth design has a crank disc, which in this embodiment, is the knurled thumb wheel (550) illustrated in FIGS. 19A-B. This crank disc (550) rotates vertically on a fixed shaft (551), with a connecting rod (553) attached via a crank pin (552) at a radius from the center. In FIG. 19B, the rod's other end (554) connects to the needle valve plunger with a pinned joint, driving the plunger (6) linearly to adjust flow through the valve body. In this design, the centroid of the crank disc is aligned with the plunger axis. This implies:
The crank disc (550) rotates about a pivot point (shaft) that is coaxial with the plunger's linear motion path.
The connecting rod (linkage) (553) attaches to the disc at an offset point (crank pin) (552) and to the plunger at its end (554), converting the disc's rotation into linear plunger motion.
The plunger shaft (18) moves linearly along its axis as the disc (550) rotates, with the displacement determined by the crank pin's radius and angular position. The motion is sinusoidal if the disc is circular.

This design presents simple kinematics: The plunger's motion is directly tied to the disc's rotation, with minimal lateral forces. However, the vertical height of the design may be less desirable for a truly compact valve body as the crank disc sits entirely above the needle valve plunger.
The disc rotates about its centroid, aligned with the plunger's axis.
A crank pin at radius r from the centroid connects to a rod, which pivots at the plunger.
Rotating the disc by angle θ moves the plunger by x=r·sin(θ) (assuming a simple pinned joint).

In another embodiment, the centroid of the crank disc may not align with the plunger axis. The mechanism can still function, but the design must account for the offset.
The crank disc rotates about a pivot point offset from the plunger's axis by some distance (e.g., horizontally or vertically displaced).
A connecting rod links the crank pin on the disc to the plunger, typically with pivot joints at both ends to allow rotational freedom.
As the disc rotates, the crank pin's circular motion is transmitted through the rod, driving the plunger linearly along its axis.

Although the plunger's linear displacement is still driven by the crank pin's position, the offset introduces a more complex kinematic relationship. The plunger's motion remains approximately sinusoidal but may be asymmetric or have a different amplitude depending on the geometry. The connecting rod's angle and length must be designed to ensure the plunger moves smoothly without binding or excessive lateral forces.

Here is the mathematical description:

```
TABLE 1
Let the disc's pivot be at (x_{d}, y_{d}), the plunger's axis along x = xₚ, and the crank pin at
radius r from the disc's centroid.
For disc rotation angle θ, the crank pin's position is:
    x_{c} = x_{d} + r · cos(θ), y_{c} = y_{d} + r · sin(θ)
The connecting rod (length L) links the crank pin to the plunger at (xₚ, yₚ), where yₚ is the
plunger's position along its axis.
The plunger's position yₚ is found by solving the kinematic constraint (distance between
crank pin and plunger):
      (x_{c} − xₚ)² + (y_{c} − yₚ)² = L²
Solving for yₚ:
  yₚ = y_{d} + r · sin(θ) ± {square root over (L² − (x_{d} + r · cos(θ) − xₚ)²)}
(The sign depends on the rod's orientation.)
This equation shows that the plunger's motion depends on the offset (x_{d} − xₚ), rod length
L, and crank radius r.
```

An advantage of the offset is that the crank disc can be positioned for a more compact valve body over the vertically aligned design. However, there are challenges to be overcome. The offset introduces lateral forces on the plunger, which may cause binding or wear unless mitigated, e.g., with a slotted or pivoting joint. Also, the connecting rod may need additional degrees of freedom, e.g., a sliding slot or longer length, to prevent jamming. Introduction of non-linearity with the plunger's motion could be optimized to support more precise flow at the low flow side of the needle plungers' travel to support precision IV therapy. Overall, the space requirements may result in a larger footprint, which is less ideal for a compact medical device.

To implement the non-aligned crank disc centroid with the valve plunger's axis, pivoting joints pinned at both ends of the connecting rod would allow rotational freedom and reduce lateral forces on the plunger. A slotted connection where a slot in the plunger's attachment absorbs lateral motion from the crank pin's arc would ensure the plunger moves only linearly. A longer rod reduces the angle of misalignment, smoothing the plunger's motion but increasing the mechanism's size.

For a needle valve in IV infusion therapy, both aligned and misaligned crank disc designs are feasible, but the choice depends on the application. The aligned design offers simplicity, and precision. It's ideal for manual thumb wheel adjustments in portable IV flow regulators, where the disc can be integrated directly above the valve. If spatial constraints or ergonomic factors require the thumb wheel to be offset, this requires careful design to maintain the fine control needed for low flow rates (e.g., 10-100 mL/hr) and to avoid binding under low-pressure conditions (<50 psi).

For a misaligned design, a thumb wheel (crank disc) is mounted 10 mm horizontally offset from the plunger's axis. A 15 mm connecting rod links a crank pin (radius 5 mm) to the plunger via pinned joints. Rotating the disc moves the plunger up to +5 mm (depending on geometry), adjusting the needle valve's orifice to control IV fluid flow. A linear guide ensures the plunger moves only vertically, preventing lateral forces from the offset.

All of these designs except for the crank disc are compact and allow rapid, single-motion adjustment of flow, making them suitable for applications requiring quick response, such as in IV infusions or laboratory setups.

The valve's flow coefficient (Cv) varies with the needle's position, typically ranging from 0 (fully closed) to a maximum of 0.5-1.0 for small needle valves (e.g., ¼″ ports or smaller). In each design, the lever's range of motion is often limited by stops on the valve body to prevent over-extension of the needle, which could damage the seat or fully retract the needle beyond the packing gland.

### Addition of Low-Cost Check Valves on the Needle Valve Outlet

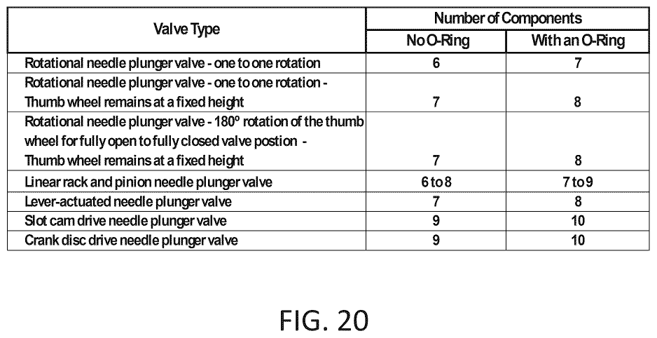

Low-cost check valves for IV therapy, including inline diaphragm, barbed, press-in, duckbill, umbrella, and disposable miniature designs, complement these five needle plunger valves by preventing backflow and ensuring safety in low-pressure (<50 psi) systems. These valves offer cracking pressures of 0.1-20 psi, biocompatible materials (USP Class VI), and scalable production methods like injection molding, making them cost-effective for disposable IV setups. They pair seamlessly with the design presented, enhancing precision and reliability in IV infusion through catheters or cannulas. The check valve options are listed in FIG. 20 with a comparison of cracking pressure, material options, and cost factors.

The most common check valves are simple elastomeric elements that can be captured on the outlet of the plastic housing with the addition of only one fitting. As shown in FIGS. 20A through 20C, a duckbill, dome, and umbrella valve element would sit between the valve housing and the additional fitting that would have a matching tubing fitting as that of the inlet on the valve.

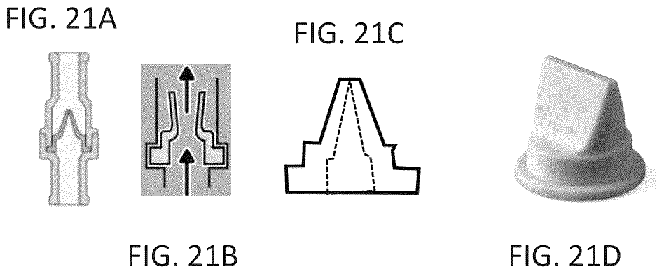

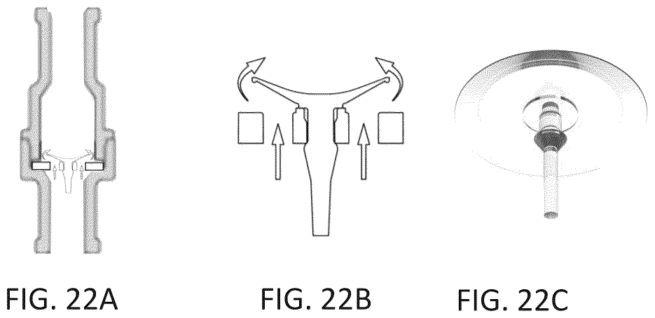

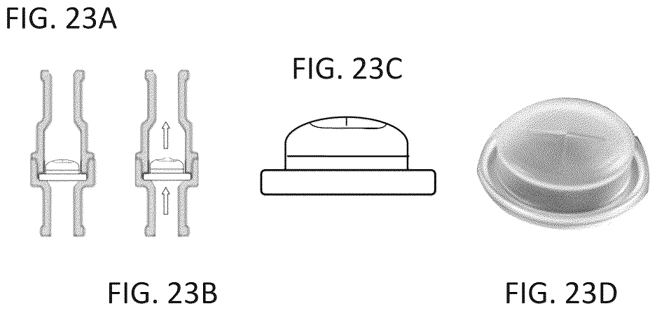

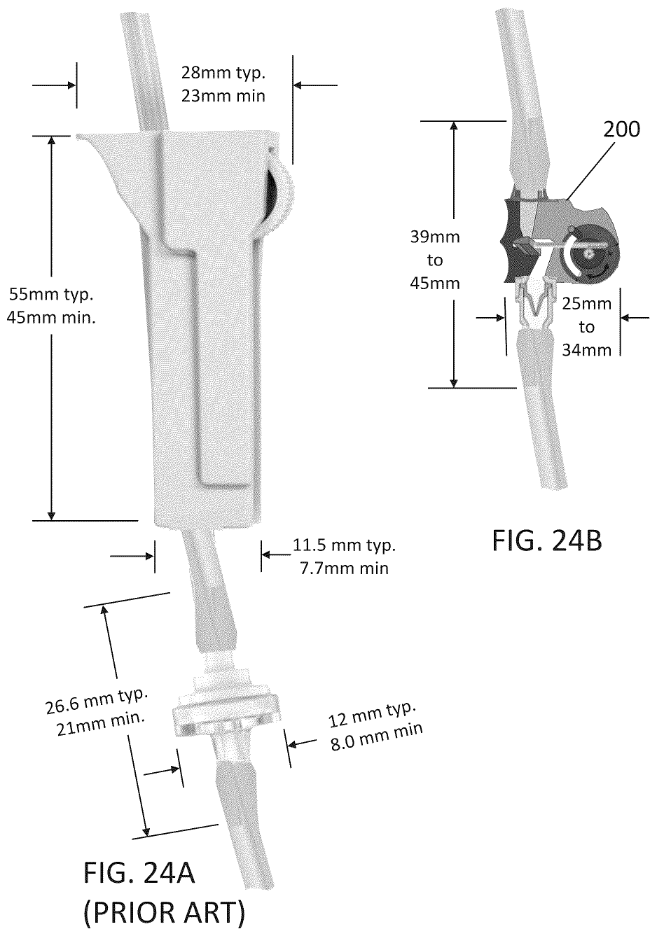

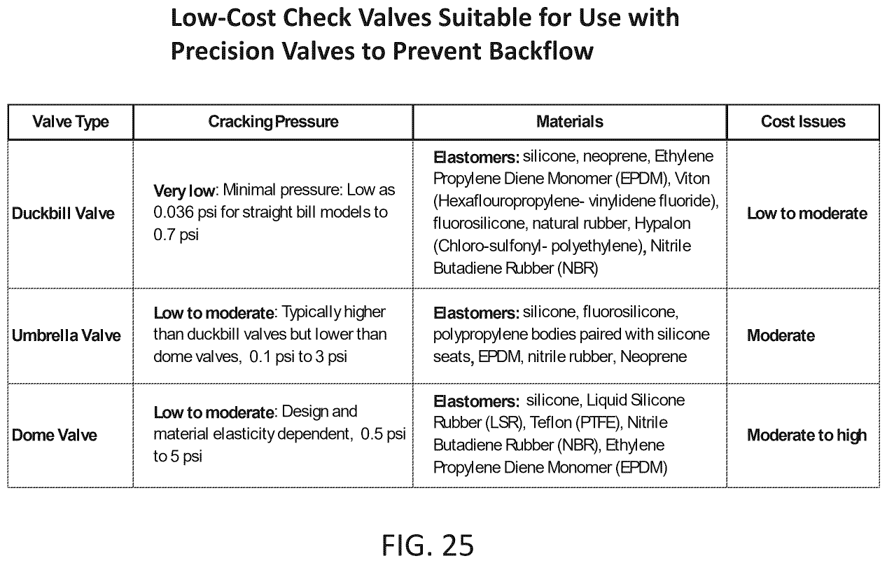

Shown in FIG. 21 is a comparison between adding a check valve in line with a roller clamp versus having the check valve as an integral part of the precision flow valve. The overall size reduction is considerable. “With only an additional molded fitting to retain the duckbill, dome, or umbrella elastomeric check valve element, the overall increase in size of the precision flow valve is minimal.

In applications where backflow cannot be tolerated, incorporation of the appropriate check valve on the outflow side of the needle plunger valve provides the desired safety factor. The small forward closure force with a check valve in line after a needle valve can affect the drip rate in a drip chamber, especially for low drip rates. The check valve introduces additional resistance to the flow as a certain minimum pressure is necessary to open. With minimum opening pressures, typically ranging from 0.4 to 1 psi for some configurations, the choice of the check valve element should match with the needle plunger valve system to ensure that precision flow control is not compromised.

For low drip rates, the pressure differential created by the needle valve might be close to the cracking pressure of the check valve. This can lead to:
1. Inconsistent drip rates: The check valve may not open smoothly at very low pressures, causing irregular dripping.
2. Reduced flow: Some of the pressure drop occurs across the check valve, potentially reducing the overall flow rate.
3. Increased sensitivity: Small adjustments to the needle valve may have a more pronounced effect on the drip rate due to the additional resistance of the check valve.

To mitigate these effects, use of the appropriate check valve type that delivers a lower cracking pressure is desirable. Manually adjusting the needle valve to precisely set a drip rate will compensate for the additional pressure drop across the check valve. It's important to note that while the check valve may introduce some challenges, it serves the crucial function of preventing backflow, which is essential in many medical and industrial applications.
1. The addition of a check valve to an IV system can affect the priming volume of the drip chamber, though the impact is generally minimal. Here's how it changes: Increased priming volume: The check valve itself adds a small amount of volume to the overall system, slightly increasing the total priming volume2.
2. Potential for air trapping: Check valves can sometimes trap small air bubbles, which may require additional priming to fully remove. This could lead to a marginal increase in the effective priming volume.
3. Flow resistance: The check valve introduces additional resistance to the flow, which may require slight adjustments to the priming process. This could result in a need for more careful filling of the drip chamber to ensure proper priming.
4. The check valve can help maintain the proper fluid level in the drip chamber once primed.

It's important to note that while these changes occur, they are generally minor and do not significantly alter the overall priming procedure. The primary focus during priming remains preventing air from entering the IV tubing and ensuring the drip chamber is filled to one-third to one-half full, according to the Clinical Procedures for Safer Patient Care Copyright @ 2015 by British Columbia Institute of Technology (BCIT).

While the check valve itself doesn't change the recommended operable liquid height, it can contribute to more stable and controlled fluid dynamics within the IV system, potentially improving the overall functionality and reliability of the drip chamber.

### Description of Needle Valve Flow Characteristics

The flow through a needle valve increases with the square of the needle's lift due to the fundamental principles of fluid dynamics and the valve's unique geometry, which together provide smooth and progressive control over liquid flow rates. A rotating needle valve consists of a slender, tapered needle that fits into a correspondingly small, conical seat within the valve body. As the needle is lifted—typically by turning a threaded stem connected to a handle or knob—the annular space between the needle and the seat increases, allowing fluid to pass through. This annular flow area is the critical factor governing the valve's flow coefficient (Cv), which quantifies the flow capacity.

The relationship between needle lift and flow arises from the geometry of the annular opening. At small lifts, the clearance between the needle and seat forms a narrow, ring-like passage. The effective flow area of this annulus is approximately proportional to the needle's lift (h) multiplied by the circumference of the needle at that point. However, because the needle is tapered, the radius of the annulus increases linearly with lift, and the flow area scales with the product of this radius and the lift. For a simplified conical needle, the flow area (A) can be expressed as A≈k*h², where k is a constant dependent on the taper angle and seat dimensions. In fluid dynamics, the flow rate (Q) through an orifice under constant pressure drop is proportional to the flow area times the square root of the pressure differential (Q=Cv*√ΔP), per the standard flow equation. Since Cv itself is directly related to the flow area, and the area increases with h², the flow rate through the needle valve effectively scales with the square of the needle lift (Q∝h²) under laminar or transitional flow conditions typical of low-pressure IV systems (below 50 psi).

This quadratic relationship offers distinct advantages for precision control, particularly in managing drip rates in IV drip chambers. At low needle lifts (e.g., 0-25% of maximum lift), the flow increases gradually because the incremental change in area—and thus flow—is small, allowing fine adjustments to achieve low drip rates (e.g., 5-10 drops per minute). As the needle is lifted further (e.g., 50-75% of maximum), the flow accelerates more rapidly due to the h² term, providing a broader range of control up to higher rates (e.g., 60-100 drops per minute) without requiring excessive travel. This smooth, progressive response contrasts sharply with the erratic behavior of roller clamps, where flow changes unpredictably due to tubing deformation and material variability. A needle valve's threaded adjustment, geared design, lever-activated, or cam slot driven mechanism further enhances this precision, as small rotational inputs translate into minute lift increments. These minute lift increments enable operators to tune the valve to exact flow rates with minimal effort.

The quadratic flow increase (Q∝h²) assumes laminar flow and a simplified conical geometry, which is typical for needle valves in low-pressure applications like IV systems. In practice, turbulence or complex taper profiles might slightly modify this, but the principle holds for design intent.

Moreover, the needle valve's design ensures repeatability and stability. Unlike roller clamps, which suffer from tubing elasticity, compression set, and plastic housing tolerances, the rigid metal or high-strength polymer components of a needle valve maintain consistent geometry across operations. This eliminates drift in flow settings over time, a common issue with roller clamps subject to material creep. The result is a valve that delivers a predictable, controllable flow profile—starting with a shallow increase at low lifts for fine-tuning, then transitioning to a steeper rise for higher flows—all within a simple, minimal-component system ideal for cost-effective replacement of roller clamps in IV fluid management.

For flow through a needle valve under low-pressure conditions (less than 50 psi), simplified equations based on the assumptions of laminar flow, incompressible fluid (e.g., water or saline in IV therapy), and steady-state conditions are presented here. Needle valves control flow by adjusting the effective orifice area, and the flow rate depends on the pressure drop, valve geometry, and fluid properties. Below is a simple set of equations for low-pressure flow through a needle valve.

### Assumptions:

Low pressure (<50 psi): Ensures negligible compressibility effects for liquids.
Laminar flow: Common in small-diameter needle valves used in IV therapy or similar applications (Reynolds number <2000).
Incompressible fluid: Typical for liquids like water or saline.
Steady-state flow: No significant transients.

Small valve opening: Needle valves often operate with a partially open orifice.

### Key Equations:

1) Volumetric Flow Rate (Hagen-Poiseuille Equation for Laminar Flow) For laminar flow through a small, cylindrical orifice or annular gap in a needle valve, the flow rate Q can be approximated using the Hagen-Poiseuille equation, adapted for the valve's effective flow path:

TABLE 2
$Q = \frac{π {r}^{4} Δ P}{8 μ L}$
• Q: Volumetric flow rate (m³/s)
• r: Effective radius of the flow path (m), determined by the valve opening
• ΔP: Pressure drop across the valve (Pa, where 1 psi ≈ 6895 Pa)
• μ: Dynamic viscosity of the fluid (Pa·s, e.g., ~0.001 Pa·s for water at 20 °C)
• L: Effective length of the flow path (m, typically short in needle valves)

Note: The effective radius r depends on the needle valve's position (how far the plunger is withdrawn). For a partially open valve, r is small, and precise values require valve-specific data or calibration.
2) Simplified Flow Rate (Orifice Flow Model)

For a needle valve treated as a variable orifice, the flow rate can be approximated using the orifice equation, adjusted for low-pressure laminar conditions:

TABLE 3
$Q = {C}_{d} A \sqrt{\frac{2 Δ P}{ρ}}$
• C_{d}: Discharge coefficient (dimensionless, typically 0.6-0.8 for needle valves,
accounts for losses)
• A: Effective orifice area (m²), proportional to valve opening
• ΔP: Pressure drop (Pa)
• ρ: Fluid density (kg/m³, e.g., ~1000 kg/m³ for water)
The area A is a function of the valve’s geometry and opening, often approximated
as A = πr² for a circular orifice or an annular area for a needle valve.

3) Valve Flow Coefficient (Cv)

In engineering practice, needle valves are often characterized by a flow coefficient Cv, which relates flow rate to pressure drop for water at standard conditions:

TABLE 4
$Q = {C}_{v} \sqrt{\frac{Δ P}{S G}}$
• Q: Flow rate (gpm, gallons per minute)
• Cᵥ: Valve flow coefficient (gpm/psi\hat{ }0.5, provided by manufacturer or experimentally
determined)
• ΔP: Pressure drop (psi)
• SG: Specific gravity of the fluid (dimensionless, ~1 for water)
For low-pressure systems, Cᵥ is small for needle valves due to their fine control.
Convert Q to SI units (m³/s) if needed: 1 gpm ≈ 6.309 × 10⁻⁵ m³/s.

For a needle valve in an IV system with:

TABLE 5
• ΔP = 2 psi ≈ 13790 Pa
• Effective radius r = 0.1 mm = 0.0001 m
• Effective length L = 1 mm = 0.001 m
• Viscosity μ = 0.001 Pa\cdotps (water/saline)
Using the Hagen-Poiseuille equation:
$Q = \frac{{π ( 0.0001 )}^{4} \cdot 13790}{8 \cdot 0.001 \cdot 0.001} = \frac{π \cdot {10}^{- 16} \cdot 13790}{8 \cdot {10}^{- 6}} \approx 5.4 \times {10}^{- 10} {m}^{3} / s$
Convert to mL/hr:
5.4 × 10⁻¹⁰ · 10⁶ · 3600 ≈ 1.94 mL/hr

This flow rate is reasonable for fine control in IV therapy, but actual values depend on the valve's specific geometry.

The needle valve design choice is well-suited for a low-pressure system such as IV fluid flows where the needle-shaped plunger allows for gradual and accurate control of fluids. By allowing for careful adjustment of the orifice size, effective pressure control in low-pressure systems can be realized.

### CONCLUSION

The core element to this set of needle valve embodiments is the geometry of the needle plunger that mates with the valve seat for full closure. This intertwined geometry enhances the level of flow control at very low flow rates and optimizes full flow when in the fully open position.

The present invention encompasses five innovative needle plunger valve designs—rotational, linear rack and pinion, lever-actuated, slot cam drive, and crank disc drive—developed to provide precise fluid flow control in low-pressure medical applications, particularly intravenous (IV) infusion therapy. Each design uniquely converts user input into linear plunger motion to adjust a valve orifice, enabling flow rates suitable for IV catheters or cannulas (e.g., 10-100 mL/hr at pressures below 50 psi). The rotational valve offers fine adjustments via a threaded mechanism, the rack and pinion design supports rapid actuation, the lever-actuated valve provides ergonomic operation, the slot cam drive allows customizable flow profiles, and the crank disc drive accommodates both aligned and offset pivot configurations for flexible integration. These designs collectively offer biocompatibility, reliability, and adaptability for manual or automated IV systems, addressing diverse clinical needs while ensuring ease of use and precision. Their compact construction, low-cost, and robust materials support sterilization and precision flow control, establishing them as ideal choices for medical environments, especially with IV infusion therapy.

FIG. 20 contains a summary of the number of components needed in each of the five innovative needle plunger valve designs described above. In counting the components in this table, the valve body is counted as two components, because it will be necessary to have an upper and a lower component due to molding issues; the remaining components are explained in the Figures and the detailed description above. A key point is that each of these designs can be produced for a much lower cost than traditional needle valves made of metal components which are used in high pressure environments.

Although the foregoing detailed description is illustrative of preferred embodiments of the present invention, it is to be understood that additional embodiments thereof will be obvious to those skilled in the art. Further modifications are also possible in alternative embodiments without departing from the inventive concepts already described.

Accordingly, it will be readily apparent to those skilled in the art that still further changes and modifications in the actual concepts described herein can readily be made without departing from the spirit and scope of the described inventions.

What is claimed is:

1: A process for precision control of a fluid in an IV delivery system, comprising controlling flow of the fluid from a first flexible tubing through a valve body to a second flexible tubing, the first flexible tubing being connected to an inlet of the valve body, the second flexible tubing being connected to an outlet of the valve body, wherein flow of the fluid through a fluid passage connecting the inlet to the outlet is controlled through use of a needle plunger disposed within the valve body, configured to adjustably restrict the fluid passage.

2: The process of claim 1, wherein control of flow of the fluid though the fluid passage by the needle plunger is controlled by rotation of a thumb wheel coupled to the needle plunger, wherein rotation of the thumbwheel translates the needle plunger to provide precise, repeatable flow control, and said flow can be adjusted between a fully open flow position and a fully closed flow position by rotation of the thumb wheel of approximately one-hundred eighty degrees or less.

3: The process of 2 wherein a height of the thumb wheel relative to the valve body remains constant between the fully open flow position and the fully closed flow position.

4: The process of 2, wherein movement between the fully open flow position and the fully closed flow position is controlled by a linear rack and pinion system operatively coupled to the needle plunger, wherein linear movement of the rack translates the needle plunger to provide precise flow control, wherein the actuation mechanism is configured for single-handled operation.

5: The process of claim 1, wherein control of flow of the fluid though the fluid passage by the needle valve is controlled by a lever actuated mechanism pivotally coupled to the valve body and operatively connected to the needle plunger, wherein linear movement of the rack translates the needle plunger to provide precise flow control.

6: The process of claim 1, wherein control of flow of the fluid though the fluid passage by the needle valve is controlled by a slot cam drive mechanism coupled to the needle plunger, wherein rotating a slot cam drive forces a pin riding in the slot of the slot cam to guide an attached needle plunger to provide customizable flow control.

7: The process of claim 1, wherein control of flow of the fluid though the fluid passage by the needle valve is controlled by a crank disc drive mechanism operatively coupled to the needle plunger, wherein rotation of the crank disc translates to upward and downward motion of the needle valve plunger to provide precise flow control.

8: A process for controlling fluid flow with a precision needle valve, comprising:
providing a needle valve comprising a valve body with a fluid passage, a needle plunger, and an actuation mechanism selected from the group consisting of rotational, linear rack and pinion, lever-actuated, slot cam drive, and crank disc drive mechanisms;
adjusting the actuation mechanism to translate the needle plunger within the valve body, thereby precisely controlling fluid flow through the fluid passage; and
preventing backflow through the valve using an integrated check valve at the outlet;
wherein the needle valve is configured for single-handed operation.

9: An intravenous (IV) infusion system, comprising:
a fluid reservoir configured to hold a medical fluid;
a tubing set connected to the fluid reservoir; and
a needle valve connected to the tubing set, the needle valve comprising:
(1) a valve body with an inlet, an outlet, and a fluid passage for permitting flow between the inlet and the outlet;
(2) a needle plunger to regulate flow through the fluid passage;
(3) an actuation mechanism coupled to the needle plunger, selected from the group consisting of rotational, linear rack and pinion, lever-actuated, slot cam drive, and crank disc drive mechanisms; and
(4) a check valve at the outlet to prevent backflow.
